(19) United States
(12) Patent Application Publication
(10) Pub. No.: US 2009/0137925 A1
(43) Pub. Date: May 28, 2009

(54) Impedance Spectroscopy Cervix Scanning Apparatus and Method

(76) Inventor: Divya Cantor, Louisville, KY (US)

Correspondence Address: MIDDLETON & REUTLINGER, 2500 BROWN & WILLIAMSON TOWER, LOUISVILLE, KY, 40202, US

(21) Appl. No.: 11/944,490

(22) Filed: Nov. 23, 2007

(51) Int. Cl. A61B 5/053; A61B 17/42

(52) U.S. Cl. 600/547; 600/591

(57) ABSTRACT

In one particular aspect of the present invention, an electrode clip is provided. The clip has a pair of lobes having opposing surfaces, and a stem extending from a side of each of the pair of lobes. A pair of tetra-pole electrodes is provided on each of the opposing surfaces. A resilient connection member joins the stems and is adapted to maintain electrical contact between the pair of tetra-pole electrodes and an actively dilating and effacing cervix.

### FIELD OF THE INVENTION

The invention pertains to impedance spectroscopy and more particularly to the use of impedance spectroscopy to measure cervical dilation and effacement.

### BACKGROUND OF THE INVENTION

In the United States, there are approximately 4,900 labor and delivery hospitals performing more than four million births annually. Of those births in US hospitals, approximately 85% of births incorporate electronic fetal monitoring during the labor course. The present invention is directed to those deliveries that receive electronic fetal monitoring.

During pregnancy, the cervix remains lengthy and thick to protect the baby. During labor, the cervix effaces and dilates. Effacement refers to the thickness of a cervix and is measured by percentage thinned. A 0% effaced cervix is thick, and a 100% is very thin. Dilation refers to the opening of the cervix and is traditionally measured in centimeters. A cervix with 0 cm dilation is closed, a cervix is in early labor between 0-4 cm, a cervix in active labor is dilated between 4 and 8 cm, a cervix in transition is between 8-10 cm, and a fully dilated cervix is approximately 10 cm. Dilation and effacement work together to open the cervix to allow the baby to pass into the vagina. The state of the cervix is only one of many critical pieces of information considered when deciding how to safely deliver the infant.

Currently, childbirth occurs with the aid of an assistant, such as a doctor, nurse, or midwife. The assistant performs a series of manual cervical examinations to monitor and assess the state of effacement and dilation of the cervix throughout labor. Labor is a lengthy process that can take several hours, up to 20 in a first time mother. During this time, the patient may be examined by more than one person.

Considering that the cervix is checked only through a manual examination, and in many cases by several assistants throughout labor, errors are inherent and inevitable. Manual cervical exams also increase the risk for infection in the mother. Furthermore, cervical exams can be uncomfortable, especially during labor contractions. Minimizing inaccuracies and increasing the quality of patient care are cornerstones for improving the healthcare system. The present invention is directed to eliminating these errors and improving patient care.

### SUMMARY OF THE INVENTION

In one particular aspect of the present invention, an electrode clip is provided. The clip has a pair of lobes having opposing surfaces, and a stem extending from a side of each of the pair of lobes. A pair of tetra-pole electrodes is provided on each of the opposing surfaces. A resilient connection member joins the stems and is adapted to maintain electrical contact between the pair of tetra-pole electrodes and an actively dilating and effacing cervix

In yet another embodiment of the present invention, an electrode assembly is provided with a clip having a pair of lobes and a stem extending from a side of each of the pair of lobes and a pair of tetra-pole electrodes provided on each of the lobes. A resilient connection member joins the stems and maintains electrical contact between the pair of tetra-pole electrodes and an actively dilating and effacing cervix. An applicator is also provided with a protrusion for engaging an opening in the clip.

In another embodiment of the present invention, an apparatus for quantifying cervical dilation and effacement is provided with a pair of tetra-pole electrodes, and a signal generating device with data collection capabilities. The pair of tetra-pole electrodes is disposed on opposing surfaces of a clip assembly. The pair of tetra-pole electrodes further maintains electrical contact between a cervix and a signal generating device. The signal generating device is capable of collecting a cervical stromal impedance value.

In still another embodiment of the present invention, an apparatus for quantifying cervical dilation and effacement is provided with a clip assembly having two opposing surfaces and a resilient connection member connecting the surfaces. A pair of tetra-pole electrodes is disposed on the pair of opposing surfaces, and a signal generating device with data collection capabilities is also provided. The clip assembly is adapted to grip a cervix and maintain electrical contact between the pair of tetra-pole electrodes and the cervix. The signal generating device is capable of collecting a cervical stromal impedance value.

In yet another aspect of the present invention, an apparatus for quantifying cervical dilation and effacement is provided. A pair of tetra-pole electrodes and a signal generating device with data collection capabilities is also provided. The pair of tetra-pole electrodes is attached to an adhesive substrate adapted to keep the pair of tetra-pole electrodes in contact with a cervix. The signal generating device is further capable of collecting a cervical stromal impedance value from the pair of tetra-pole electrodes.

In another aspect of the present invention, a method for quantifying cervical dilation and effacement of a patient's cervix is provided. A pair of tetra-pole electrodes is provided with a signal generating device with data collection capabilities. The pair of tetra-pole electrodes is adhesively affixed to a patient's cervix. The electrodes are then coupled to the signal generating device. A signal is sent from the signal generating device to the pair of electrodes. A signal from the tetra-pole electrodes is then collected. The value of this signal is the cervical stromal impedance value. A cervical dilation and effacement value is then calculated based upon the cervical stromal impedance value.

### BRIEF DESCRIPTION OF THE ILLUSTRATIONS

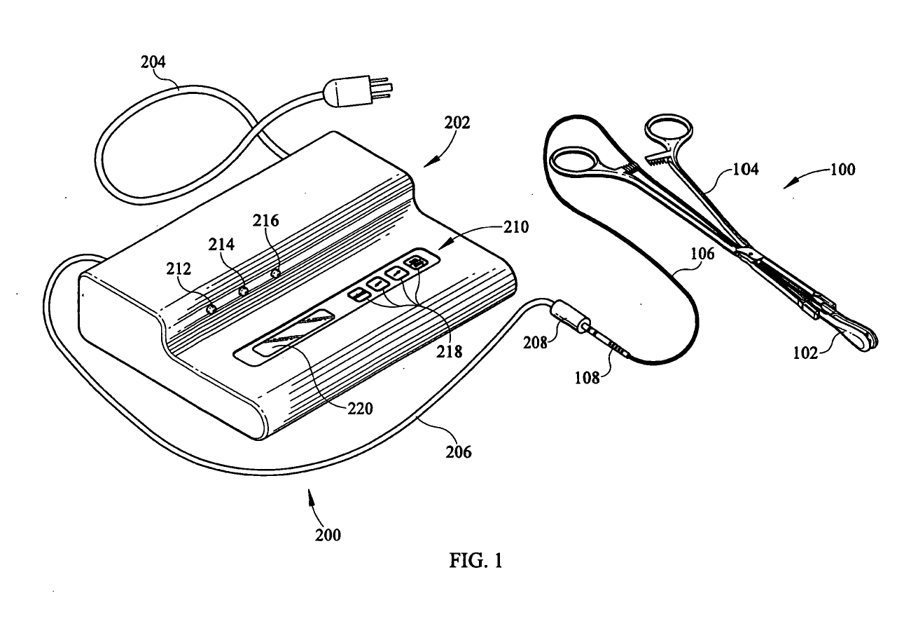

FIG. 1 is an overall view of an embodiment of the present invention.

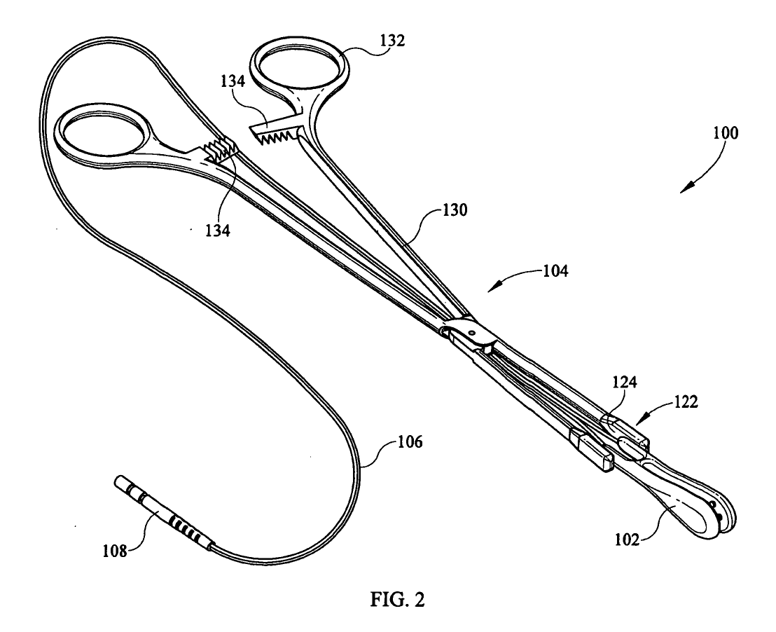

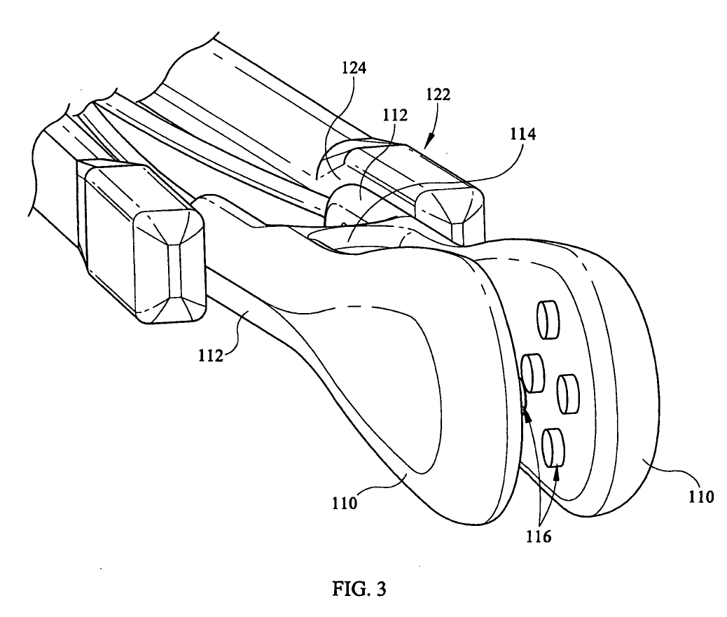

FIG. 2 is a first view an electrode clip assembly and forceps-type applicator of the present invention.

FIG. 3 is another view of the embodiment shown in FIG. 2.

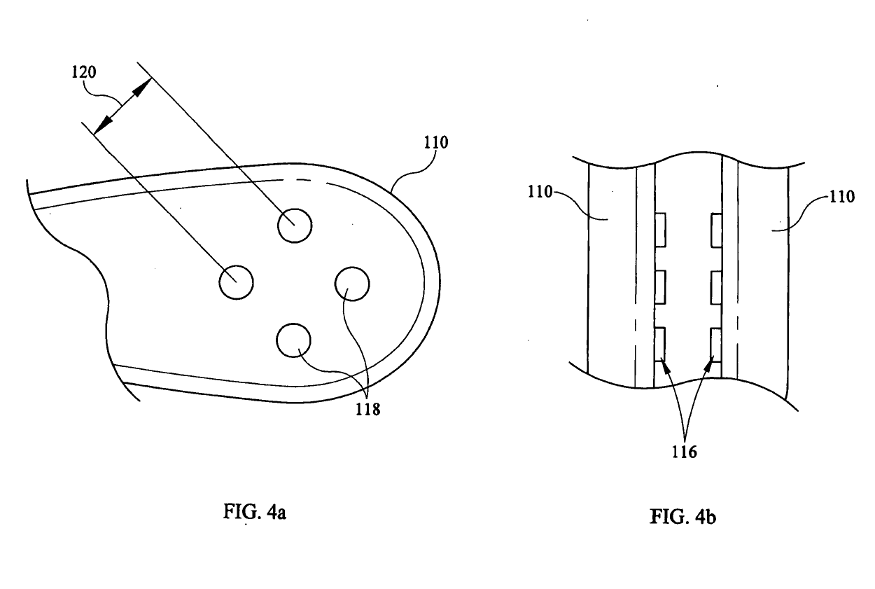

FIG. 4 illustrates a tetra-pole electrode of the present invention.

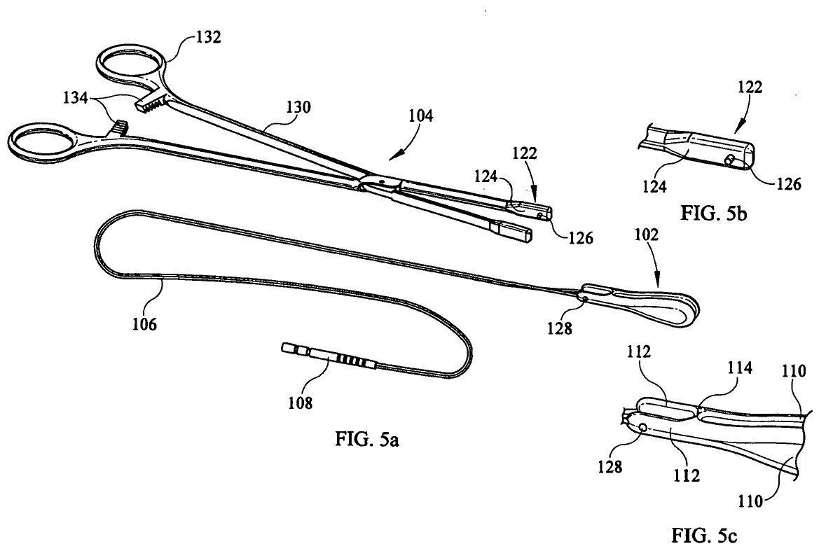

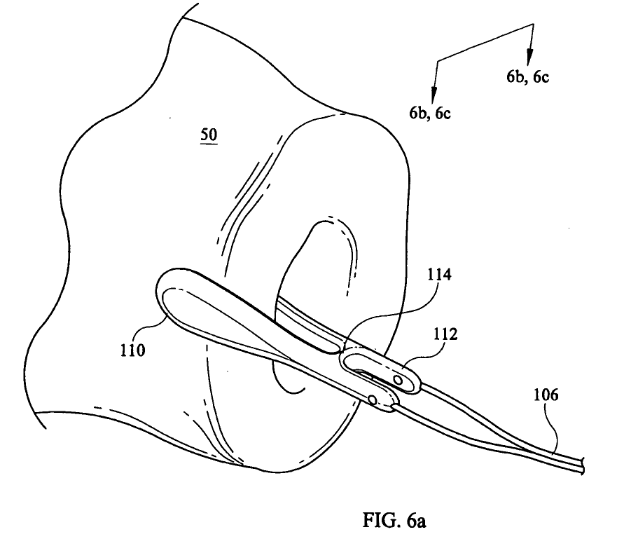

FIG. 5 is an exploded assembly view of the embodiment shown in FIG. 2.

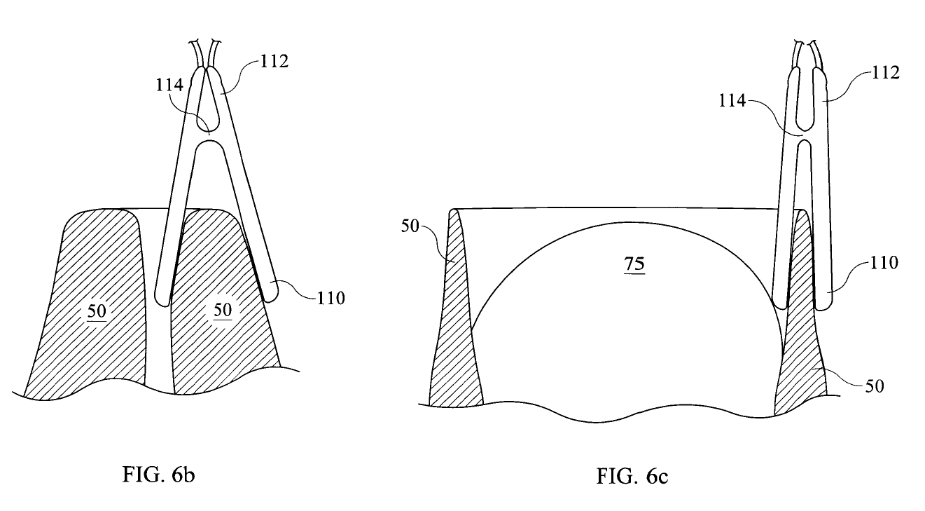

FIG. 6a shows an electrode clip assembly of the present invention placed on a cervix.

FIG. 6b is a cross-sectional view of the embodiment of FIG. 6a, shown along the line 6b-6b, showing a cervix with little to no dilation or effacement.

FIG. 6c is a cross-sectional view of the embodiment of FIG. 6a, shown along the line 6c-6c, showing a cervix with a substantial amount of dilation and effacement.

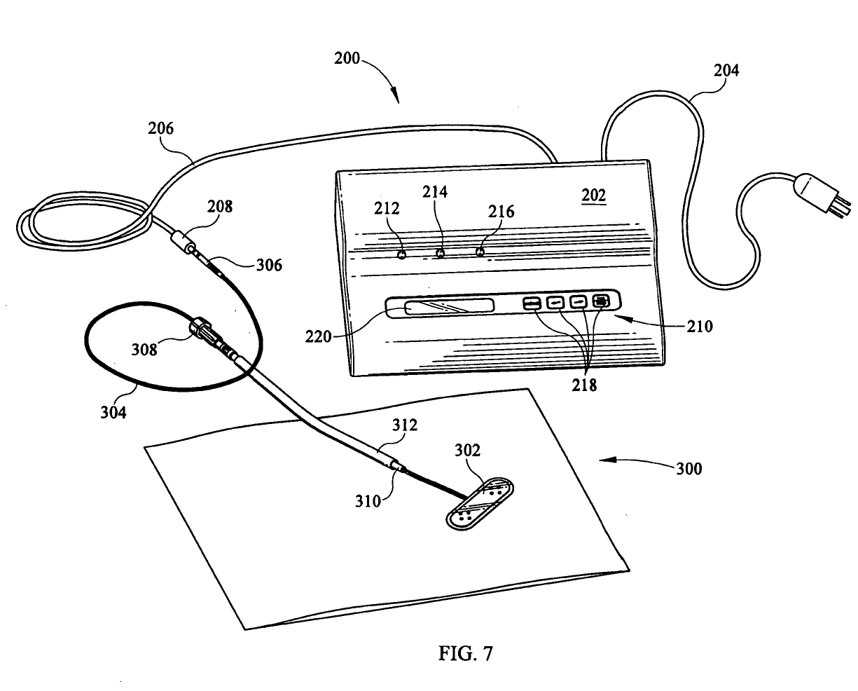

FIG. 7 is an alternative embodiment of the present invention.

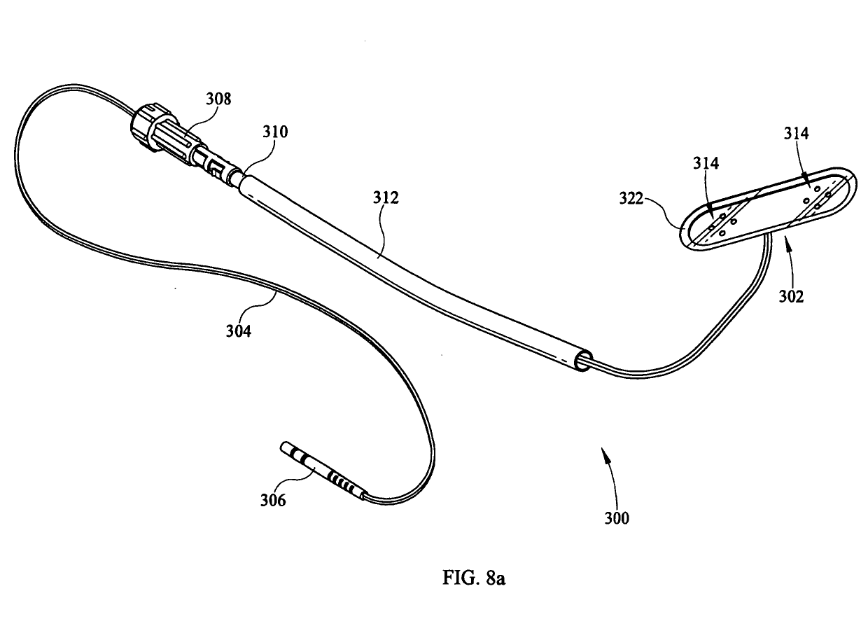

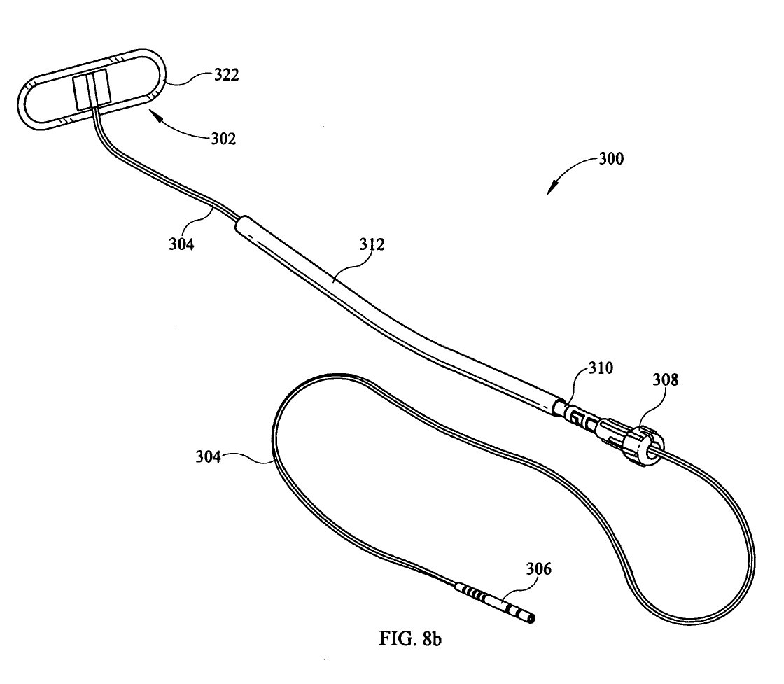

FIGS. 8a and 8b depict an electrode and adhesive substrate assembly.

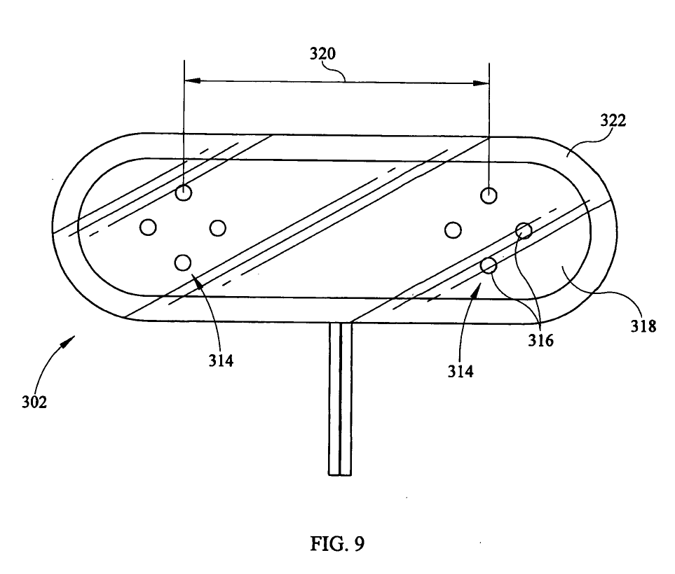

FIG. 9 illustrates a tetra-pole electrode and adhesive substrate of the present invention.

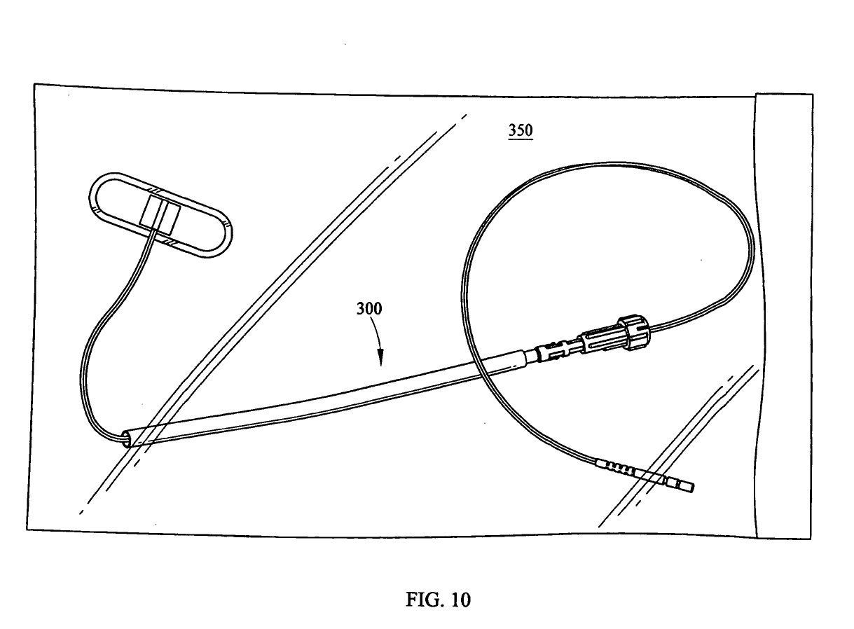

FIG. 10 shows the embodiment shown in FIGS. 8a and 8b sealed in a sterilized envelope.

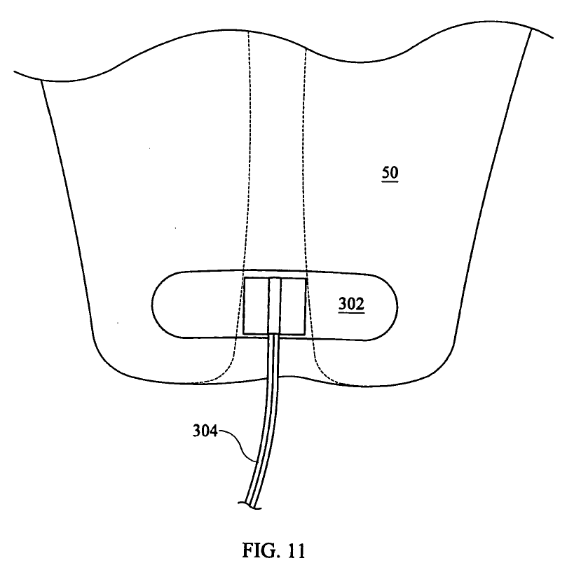

FIG. 11 depicts the tetra-pole electrode and adhesive substrate of the present invention placed on a cervix.

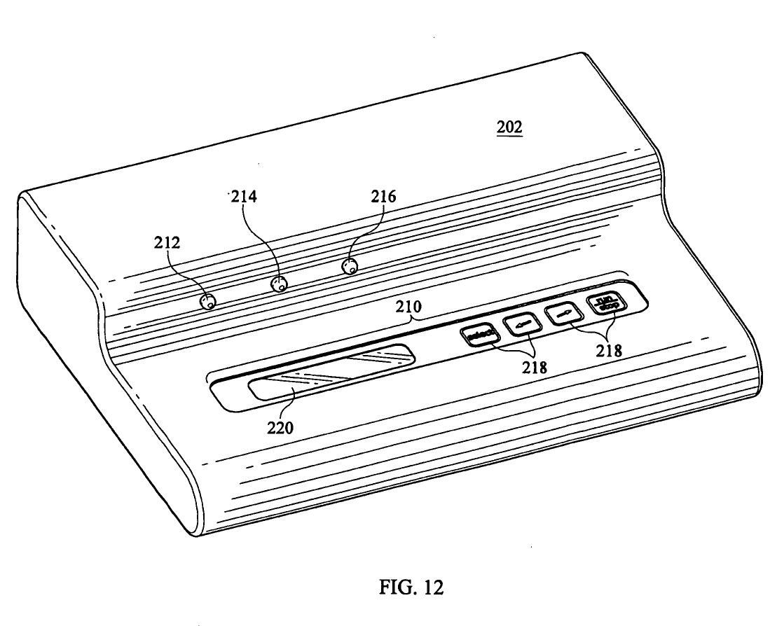

FIG. 12 shows a data acquisition unit of the present invention.

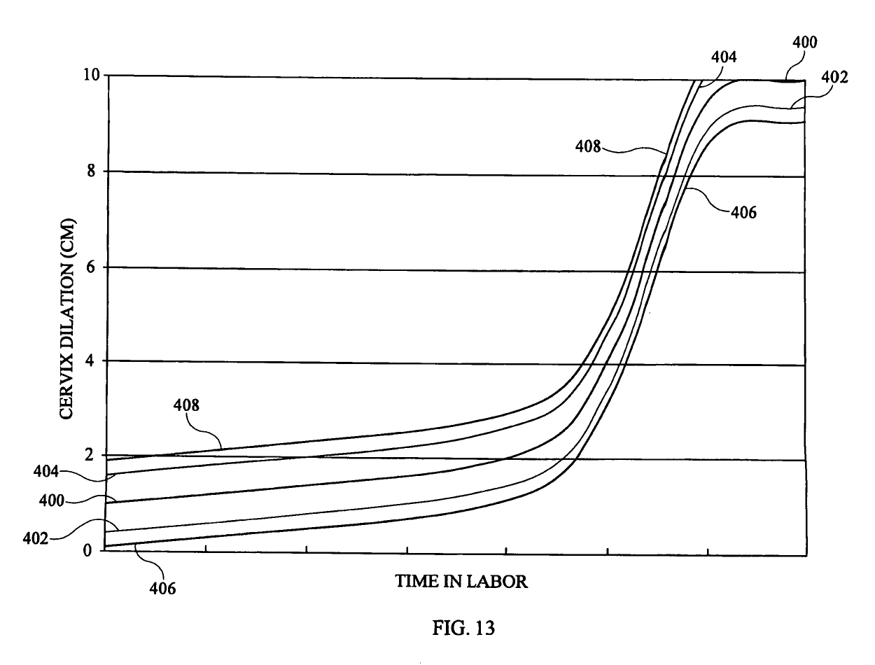

FIG. 13 depicts a normal labor curve with error curves.

### DETAILED DESCRIPTION OF THE INVENTION

The present invention provides an autonomous electronic system for continuous labor room monitoring of the uterine cervical progress during the birthing process. The dilation and effacement are the two specific changes this device will monitor. Cervical dilatation and effacement are tracked indirectly by continual measurement of the electrical impedance of the cervix, or cervical stromal impedance (CSI). Measurement of CSI has been established as a non-invasive means of assessing the cervical tissue characteristics which vary during pregnancy and labor.

Referring now to FIG. 1, a disposable, one-time use electrode and wire assembly 100, and a bench top or rack mounted monitor assembly 200 are provided.

Referring now to FIGS. 1-5, a disposable, one time use electrode and applicator assembly 100 is provided. The electrode and applicator assembly 100 has an electrode clip assembly 102, a forceps-type applicator 104, a two-wire lead 106, and a transducer connector 108. The transducer connector 108 mates with the transducer cable 206 of the monitor assembly 200. The entire disposable assembly 100 may be packaged in a sealed envelope sterilized by gamma irradiation prior to delivery to the end user.

The electrode clip assembly 102 is provided with two lobes 110 and a stem section 112 extending away from each lobe 110. The two lobes 110 and stem sections 112 may or may not be symmetric with respect to each other. The two stem sections 112 are connected by a resilient connection member 114. The entire electrode clip assembly 102 is preferably smooth and atraumatic. The resilient connection member 114 is capable of flexing, allowing the two lobes 110 to move and rotate with respect to each other. The overall length of the lobes 110 and stem sections 112 is preferably 5 cm or less. The overall length of the lobes 110, is preferably 2 cm or less and 1 cm or less in width.

Two tetra-pole electrodes 116 are positioned on opposing surfaces of the lobes 110. Each tetra-pole electrode 116 is provided with four electrode pads 118. The electrode pads 118 are approximately 1.6 mm in diameter. The pads 118 are further provided in a square tetra-pole formation. The individual pads 118 are spaced apart at a fixed distance 120 from the center of the pads 118 along the side of the tetra-pole formation. The fixed distance 120 may be 3.9 mm. The electrode pads 118 are made of any suitable conductive material that is inert within the human body, such as, for example, gold, silver, or palladium. The two-wire lead 106 is connected to the tetra-pole electrodes 116 and extend out of the electrode clip assembly 102 through the stem sections 112. The two-wire lead 106 terminates at a transducer connector 108. When installed, only the lobes 110 and tetra-pole electrodes 116 will be in contact with the cervix.

The lobes 110, stem sections 112 and resilient connection member 114 are preferably made of a material that is inert within the human body. In addition, the lobes 110, stem sections 112 and resilient connection member 114 may be formed of the same material, such as a plastic. However alternatively, the lobes 110, and stem sections 112 may be molded from a plastic and the resilient connection member 114 may be a resilient metal spring, clip or band. If the resilient connection member 114 is formed of resilient metal, then it may be molded into the stem sections 112 when they are molded. In addition, the tetra-pole electrodes 116 and the two-wire lead 106 may also be molded into the lobes 110 and stem sections 112.

One embodiment of the present invention also provides a forceps-type applicator 104. The forceps-type applicator 104 may be provided by the physician, but may preferably be provided with the electrode clip assembly 102. Though it will not be in the body for extended periods, the forceps-type applicator 104 is preferably made of a material that is inert within the human body, such as a plastic material.

The forceps-type applicator 104 is provided with jaws 122 having a gripping surface 124. The gripping surface may be provided with a protrusion 126 that engages an opening 128 on an exterior of the stem sections 112. The mating engagement of the jaw protrusion 126 and stem opening 128 secures the electrode clip assembly 102 to the forceps-type applicator 104 during installation of the electrode clip assembly 102, but does not impede removal of the forceps-type applicator 104 after installation. The opening 128 may be provided on the stem sections 112 such that it 128 is on the opposite side of the resilient connection member 114 as the lobes 110. In this configuration, when the forceps-type applicator 104 is closed, its jaws 122 and protrusion 126 press against the stem sections 112 and opening 128 such that the resilient connection member 114 acts as a fulcrum, spreading the lobes 110 apart. Opening the jaws 122, thus closes the lobes 110. To remove the jaws 122 from the stem sections 112, the forceps-type applicator 104 is simply opened until the protrusions 126 are removed from the openings 128. The protrusions 126 and openings 128 may be made of a non symmetric shape or pattern of multiple protrusions 126 and openings 128 on each respective jaw 122 and stem section 112 such that axial orientation of the electrode clip assembly 102 is maintained during the installation process.

Alternatively, the jaws 122 and gripping surface 124 may be void of any type of protrusion. For this configuration, the jaws 122 may simply slip into an opening (not illustrated) in the stem sections 110, such that the jaws 122 may be removed simply by pulling the jaws 122 away from the electrode clip assembly 102 after it has been placed on the cervix. In this configuration, spreading the jaws 122 also opens and spreads the lobes 110. Closing the jaws 122 then closes the lobes 110.

The forceps-type applicator 104 is further provided with handles 130 that may have gripping features 132, such as finger loops. In addition, the forceps-type applicator 104 may further be provided with a locking feature 134 to clamp the forceps-type applicator 104 closed.

Referring now to FIGS. 6a-6c, installation of the electrode clip assembly 102 is facilitated with the forceps-type applicator 104. The forceps-type applicator 104 is held in one hand with the electrode clip assembly 102 secured to the jaws 122 by the engagement between the protrusion 126 and the stem opening 128. In this manner, the electrode clip assembly 102 may be guided through the vaginal opening with the other hand. The forceps-type applicator 104 is then used to open the electrode clip assembly 102 as substantially described herein. The electrode and clip assembly 102 is then grasped atraumatically on the lip of the cervix that is most easily accessible at the time of application. The forceps-type applicator 104 is then used to closed the electrode and clip assembly 102 as substantially described herein, allowing the assembly 102 to gently clasp the cervix. The forceps-type applicator 104 is then removed from the electrode clip assembly 102 as substantially described herein and removed from the vaginal opening. The forceps-type applicator 104 may then be discarded.

FIG. 6b is a shows the electrode clip assembly 102 grasping the lip of a cervix 50 with little to no dilation or effacement. In FIG. 6c, the cervix lip 50 has dilated and effaced much more than that shown in FIG. 6b. However, the resilient connection member 114 maintains electrical contact between the tetra-pole electrodes 116 and the cervix 50. It is important that the electrode clip assembly 102 be atraumatic. Not only is it important not to injure the cervix but, as shown in FIG. 6c, the infant's head 75 may come into contact with the clip 102.

The transducer connector 108 is then mated with the transducer cable 206 which is connected to the data acquisition unit 202. With the electrode clip assembly 102 in place on the cervix, Cervical Stromal Impedance (CSI) is measured via the set of tetra-polar electrodes 116.

Prior to the birth of the child, the attending physician, nurse, or other caregiver will remove the electrode clip assembly 102 from the cervix. This may be accomplished by simply pulling on the two-wire lead 106, thus removing the electrode clip assembly from the vaginal opening. The electrode clip assembly 102 may then be discarded.

Referring to FIGS. 7-8b, an alternative electrode and wire assembly 300 may be provided with a bench-top or rack-mounted monitor 200. The alternative electrode and wire assembly 300 may be provided with an electrode and adhesive substrate assembly 302, a two-wire lead 304, a transducer connector 306, an end cap 308, an inner tube 310, and an outer tube 312.

Referring now to FIG. 9, the electrode and adhesive substrate assembly 302 is provided with two tetra-pole electrodes 314, each having four electrode pads 316. The electrode pads 316 are approximately 1.6 mm in diameter. The electrode pads 316 are arranged in a square tetra-pole formation spaced from centers 3.9 mm apart. The tetra-pole electrodes 314 are spaced from center at a fixed distance 320, which may be 25 mm. The electrode pads 316 are made of any suitable conductive material that is inert within the human body, such as, for example, gold, silver, or palladium. The two-wire lead 304 is connected to the tetra-pole electrodes 314.

The electrode and adhesive substrate assembly 302 is further provided with a wet-tissue adhesive substrate 318. A peel-off backing 322 is provided to protect the adhesive substrate 318 prior to use. During use, the wet-tissue adhesive substrate 318 is used to attach the electrodes 314 to maintain electrical contact with the cervix. The overall dimensions of the electrode and adhesive substrate assembly 302 are approximately 4.8 mm by 1.6 mm by 1 mm.

The two-wire lead 304 extends from the electrode and adhesive substrate assembly 302, and further extends through an end cap 308, inner tube 310, and an outer tube 312. The two-wire lead 304 terminates at a transducer connector 306. Referring now to FIG. 10, the entire disposable assembly is packaged in a sealed envelope 350 and sterilized by gamma irradiation prior to delivery to the end user.

For installation, the applicator outer tube 312 is held in one hand, the peel-off backing 322 is removed from the adhesive substrate 318. Grasping the outer tube 312, the electrode and adhesive substrate assembly 302 is affixed to the anterior lip of the cervix 50 with the index and middle fingers using gentle pressure, as shown in FIG. 11. The inner tube 110, outer tube 112, and end cap 308 are removed by sliding along the two-wire lead 304 and over the transducer connector 306.

The transducer connector 306 is then mated with the transducer cable 206 which is connected to the data acquisition unit 202. With the electrode assembly in place on the cervix, CSI is measured via the small set of tetra-polar electrodes 314.

Referring to FIGS. 1, 7 and 12, a monitor assembly 200 is provided with data acquisition unit 202, a power cord 204, and a transducer cable 206. The monitor 200 is preferably connected to a mains electric source by the power cord 204, but a battery electric source may also be utilized. The transducer cable 206 mates with the transducer connector 108, 306. The transducer cable 204 includes analog-to-digital signal conditioning hardware 208 in a small enclosure at the patient-end of the cable 206. The data acquisition unit is provided with a power supply, a signal generator, signal analysis hardware and software, and a user interface 210. The data acquisition unit 202 is provided with a low voltage, low current AC amplitude comparator circuit including a load resistor. The load resistor preferably has an impedance value that closely matches the magnitude of typical CSI values. CSI values are measured in Ohm-m. Typical values may be found in Gandhi, et al., “Comparison of Human Uterine Cervical Electrical Impedance Measurements Derived Using Two Tetrapolar Probes of Different Sizes,” European Journal of Obstetrics & Gynecology and Reproductive Biology, Vol. 129, Issue 2, December 2006, 145-149, which is incorporated herein by reference in its entirety. The monitor assembly 200 may output a preset fixed or user input variable signal.

The assembly 200 may measure the CSI of the cervix at a preset fixed or user input variable frequency, such as, for instance once every minute. The assembly 200 may also be provided with the capability of storing a fixed preset or user input variable amount of data, such as, for instance, 24 hours of readings. This data may also be downloaded to a text or excel file at the user's discretion.

The user interface 210 is located on a front panel of the data acquisition unit 202 and is provided with LEDs 212, 214, 216, membrane switches 218, and a display screen 220. The LEDs comprise a green LED 212, an amber LED 214, and a red LED 216, the operation of which is explained in greater detail herein. The membrane switches are used to input various data, personalized for each patient. Such data is explained in greater detail herein. The display screen 220 is provided to confirm entered data, as well as provide a visual output for data, such as, for example, current or past CSI values.

When a patient first comes to the hospital and is admitted to labor and delivery, the electrode and clip assembly is installed as described herein, and a baseline CSI value is measured. An initial manual cervical exam may also be given to determine a baseline cervical dilation and effacement value.

As labor progresses, CSI values are continuously measured, recorded, and monitored at a specific rate, such as once per minute. Control software with an analyzing algorithm is used to interpret the measured CSI values. The algorithm may consider a variety of data to properly analyze the CSI, such as, for example, the current CSI value, previously measured CSI values, including the baseline CSI value, the baseline cervical dilation and effacement value, the time since the baseline CSI value or cervical dilation and effacement value was measured, the age, weight, number of pregnancies, number of vaginal births, or any other relevant data of the mother, a database of empirical data that contains similar data as well as actual CSI values and corresponding cervical dilation and effacement values. The algorithm may use any of the data described herein to extrapolate a cervical dilation and effacement value from the measured CSI value. Thus, eliminating the need for manual cervical exams during labor.

As described herein, the algorithm may utilize a database of empirical data containing actual CSI values and corresponding cervical dilation and effacement values. Referring now to FIG. 12, this database of empirical data may be used to plot a labor curve 400, such as a Friedman labor curve, comparing time in labor v. cervical dilation and/or effacement. Alternatively, a curve may be generated comparing time in labor v. CSI values. Labor curve 400, may represent how a normal labor should progress.

The control software and analyzing algorithm determines the difference or error of a measured CSI curve from the labor curve 400 to determine which of three front-panel LEDs 212, 214, 216 will be illuminated. The difference or error of a measured CSI curve relative to the labor curve 400 may be determined by, for example, statistical deviations from the labor curve 400 or an analysis of the slope of the measured CSI curve relative to the slope of the labor curve 400. A green LED 212 is illuminated to indicate an acceptable error relative to the labor curve 400. The green LED 212 will illuminate if the measured CSI curve falls between the inner error curves 402, 404. An amber LED 214 is indicative of a measured cervical progress rate (cm/hr) that is not following the prescribed labor curve, but still within predetermined acceptability bounds. The amber LED 214 will illuminate if the measured CSI curve falls between either of the inner error curves 402, 404, and the outer error curves 406, 408. Illumination of a red LED 216 indicates progress so at an unacceptable rate of change that the clinician is alerted to more closely assess and evaluate the patient condition. The red LED 216 will illuminate if the measured CSI curve falls outside of the outer error curves 406, 408. If the amber 214 or red 216 LED is lit, then the cervix is dilating and/or effacing either faster or slower than a prescribed set of norms. In addition to the LEDs, various auditory signals may be employs to signal attending caregivers that the cervix is dilating and/or effacing either faster or slower than a prescribed set of norms.

When the cervix has sufficiently effaced and dilated but prior to the birth of the infant, the electrode and adhesive substrate 300 may be removed from the cervix and the entire electrode and wire assembly may be removed from the birth canal.

It is anticipated that values of multiple labor curves will be stored in the firmware stored in the data acquisition unit 202 and accessed by the analyzing algorithm. These multiple labor curves may accommodate individual health parameters and factors such as, for example, gravida, para, pre-pregnancy weight, current weight, height, history of gestational Diabetes Mellitus and Type I Diabetes Mellitus. Gravida refers to the number of times a woman has been pregnant and para refers to the number of births. In this manner, the acceptable cervical progress rate threshold can be varied for each individual patient. This information is added via the membrane switches 218 on the front panel of the data acquisition unit 202.

The present invention may provide several advantages over the current state of the art. First, the patient's care may be enhanced. Secondly, the physician may provide more accurate diagnosis and finally, the hospital may have improved efficiencies in the labor and delivery unit.

A first advantage for the patient may relate to an induction birth. An induction is when a patient is brought into labor through various medicines. This is performed when a maternal-fetal condition necessitates delivery. The present invention may be able to provide detailed cervical information allowing the physician to administer the appropriate medication and treatments.

A second advantage that may be afforded to the patient relates to what is known as a missed or precipitous delivery. Some deliveries can occur very fast and the attending physician will not be given the opportunity to arrive in time for the delivery. If advanced warnings can be given when the cervix is changing rapidly, then a safer delivery can take place with the physician present.

Another advantage that may be provided to the patient concerns to a condition known a shoulder dystocia. This serious condition occurs when the shoulders of the infant are ‘stuck’ and will not deliver without certain maneuvers being performed. This condition has been known to result in both permanent and transient neurological damage and possible infant death. Measurements from the present invention may be able to alert the physicians and nurses of a prolonged labor, a risk factor for shoulder dystocia. Alerts may be instituted on the monitor if the progression of labor does not follow the expected progression.

A fourth possible benefit for the patient involves preterm labor. A preterm infant is one that delivers prior to 37 weeks gestation. Preterm births can result in lifelong developmental challenges, such as cerebral palsy, mental retardation, chronic lung disease, and vision and hearing compromises. The present invention may properly identify the patient at risk for this condition allowing the physician to tailor the treatment plan by documenting true cervical change rather than uterine contractions that do not produce cervical change. This is currently not performed; preterm labor has a ‘one size fits all’ type of treatment plan and is generally not individualized.

Finally, the patient may benefit by having a reduced number of exams at regular intervals by the care giver. A cervical examination is always an uncomfortable procedure for the mother. It is also important to recognize the potential for reduced infections when fewer manual examinations are performed. The present invention may greatly minimize the need for repetitive exams. Therefore, it is expected that this device can help lower this incident. In addition, patients may have the most up to date knowledge of their cervical changes reflecting favorably that better care is being received.

The hospitals that use the present invention may also see benefits. The present invention offers cost efficiencies to the hospital based on improved efficiencies of the nursing staff. Given that one cervical examination may take 2-3 minutes with another 5-7 minutes for data entry into the patient's medical record, the 10 minutes for this exam can be used to perform another task. If a nurse checks a patient once every hour and the nurse to patient ratio is 1:2, and an exam is performed every 2 hours on the same patient, then a savings of 10 minutes/hour over a 12 hour shift translates into 120 minutes of time to be used elsewhere. This can result in a cost efficiency of approximately $200K in a 10 bed Labor and Delivery unit per year. It is important to note that this device will not eliminate the need for a manual examination; rather the present invention would make the entire process more efficient.

While there has been described and illustrated particular embodiments of a single character printing device and method of printing, it will be apparent to those skilled in the art that variations and modifications may be possible without deviating from the broad spirit and principle of the present invention, which shall be limited solely by the scope of the claims appended hereto.

I claim:

1. An electrode clip, comprising;
a pair of lobes having opposing surfaces;
a stem extending from a side of each of said pair of lobes;
a pair of tetra-pole electrodes disposed on said pair of opposing surfaces;
said pair of lobes being pivotable with respect to each other about two or more axes; and
a resilient connection member joining said stems and adapted to maintain electrical contact between said pair of tetra-pole electrodes and an actively dilating and effacing cervix.

2. The electrode clip of claim 1, wherein said stems extend beyond said resilient connection member, away from said pair of lobes.

3. The electrode clip of claim 1, wherein said stems comprise an opening adapted to accept part of a tool to facilitate installing the clip on said cervix.

4. The electrode clip of claim 1, wherein said clip is disposable.

5. (canceled)

6. An electrode assembly, comprising;
a clip having a pair of lobes and a stem extending from a side of each of said pair of lobes and a pair of tetra-pole electrodes disposed on said pair of lobes;
said pair of lobes being pivotable and translatable with respect to one another;
a resilient connection member joining said stems
an applicator; and
wherein said applicator comprises a protrusion for engaging an opening in said clip.

7. The electrode assembly of claim 6, wherein said applicator is a pair of forceps.

8. The electrode assembly of claim 7, wherein said applicator is provided with a pair of jaws, adapted to move said stems together.

9. An apparatus for quantifying cervical dilation and effacement comprising:
a pair of tetra-pole electrodes;
a signal generating device with data collection capabilities;
wherein said pair of tetra-pole electrodes is disposed on opposing surfaces of a clip assembly;
wherein said pair of tetra-pole electrodes maintain electrical contact between a cervix and said signal generating device; and
wherein said signal generating device is capable of collecting a cervical stromal impedance value; and
wherein said pair of tetra-pole electrodes are joined via a resilient member.

10. The apparatus for quantifying cervical dilation and effacement of claim 9, wherein said signal generating device is further provided with data conditioning and collection capabilities.

11. The electrode clip of claim 1, wherein said resilient member comprises a spring.

12. (canceled)

13. (canceled)

14. The electrode assembly of claim 6, wherein said resilient member comprises a spring.

15. (canceled)

16. (canceled)

17. The apparatus for quantifying cervical dilation and effacement of claim 9, wherein said resilient member comprises a spring.

18. (canceled)

19. (canceled)

20. The electrode clip of cliam 1, wherein said lobes are translatable with respect To one another.

21. The electrode clip of claim 1, wherein said resilient member is a living hinge.

22. The electrode clip of claim 6, wherein said resilient member is a living hinge.

23. The electrode clip of claim 9, wherein said resilient member is a living hinge.
